(19) United States
(12) Patent Application Publication
(10) Pub. No.: US 2013/0037251 A1
(43) Pub. Date: Feb. 14, 2013

(54) Liquid cooled thermal control system and method for cooling an imaging detector

(71) Applicants: Ashutosh Joshi, Waukesha, WI (US); Joseph Lacey, Cambridge, WI (US); Venkatarao Ryali, Bangalore (IN)

(72) Inventors: Ashutosh Joshi, Waukesha, WI (US); Joseph Lacey, Cambridge, WI (US); Venkatarao Ryali, Bangalore (IN)

(73) Assignee: General Electric Company, Schenectady, NY (US)

(21) Appl. No.: 13/208,109

(22) Filed: Aug. 11, 2011

(51) Int. Cl. F25B 29/00; A61B 6/03

(52) U.S. Cl. 165/201; 378/19

(57) ABSTRACT

A liquid cooled thermal control system and method for cooling an imaging system are provided. One imaging system is a computed tomography (CT) system having a detector that is positioned on a detector rail. The detector includes a plurality of detector components. At least some of the detector components are configured to detect x-rays. A liquid cooled thermal control system is provided having cooling channels in thermal communication with the detector rail. The cooling channels have a cooling fluid flowing therethrough to control a temperature of the detector components in response to one or more disturbances that changes a temperature of the x-ray detector. A control module is also provided in the liquid cooled thermal control system to adjust parameters of the liquid cooled thermal control system in response to the disturbances.

### BACKGROUND OF THE INVENTION

The subject matter described herein relates generally to imaging detectors, such as computed tomography (CT) detectors, and more particularly, to a cooling system for CT detectors.

CT detectors may include a detector rail having a plurality of detector components positioned thereon. The detector components also may include a collimator having openings formed therein to direct x-rays emitted from a subject to a scintillator. The collimator separates the x-rays along the scintillator. The x-rays are then converted to light waves with a plurality of photodiodes positioned behind the scintillator. An analog-to-digital convertor converts the analog light waves to digital signals that can be generated into an image of the subject.

Generally, the detector components of the CT detector generate a considerable amount of heat. The detector components may be sensitive to the heat generated by the CT detector. For example, the heat may cause the detector components to shift on the detector rail. As such, the openings of the collimator may become misaligned with openings in the scintillator, leading to scatter or noise in an image generated by the CT detector. Additionally, some detector components are sensitive to changes in temperature. For example, the photodiodes may overheat or become damaged if exposed to large changes in temperature. This is particularly problematic given that large amounts of heat are generated by the analog-to-digital converter which is positioned adjacent to the photodiodes.

Conventional means to cool heat generated by the CT detector include cooling the detector with fans, heat sinks, or the like. However, such methods do not maintain a temperature of the CT detector, but rather, merely supply cooled air to the components. As such, temperature variations still exist within the CT detector, leading to shifting of the detector components and/or sensitivity of the components. Other CT detectors do not attempt to cool the components, but rather, compensate for heat within the detector through software. In particular, the temperature of the CT detector is monitored and data acquisition and image formation are compensated for based on the detected temperature. Such methods may be undesirable as software corrections may lead to error within the data.

### SUMMARY OF THE INVENTION

In one embodiment, a computed tomography (CT) detector is provided having a detector rail. An x-ray detector is positioned on the detector rail. The x-ray detector includes a plurality of detector components. At least some of the detector components are configured to detect x-rays. A liquid cooled thermal control system is provided having cooling channels in thermal communication with the detector rail. The cooling channels have a cooling fluid flowing therethrough to control a temperature of the detector components in response to one or more disturbances that changes a temperature of the x-ray detector. A control module is provided in the liquid cooled thermal control system to adjust parameters of the liquid cooled thermal control system in response to the disturbances.

In another embodiment, a liquid cooled thermal control system for a computed tomography (CT) detector is provided. One or more cooling channels are provided in thermal communication with a detector rail of the CT detector. The cooling channels have a cooling fluid flowing therethrough to control a temperature of detector components positioned on the detector rail in response to one or more disturbances that changes a temperature of the detector rail. A heat exchanger is provided for receiving heated cooling fluid from the cooling channels. The heat exchanger cools the cooling fluid. A heater is also provided for receiving the cooled cooling fluid from the heat exchanger. The heater heats the cooled cooling fluid from the heat exchanger and discharges the cooling fluid into the cooling channels. A control module is provided for controlling at least one of the heat exchanger, the heater, or a fan of the heat exchanger to control a temperature of the cooling fluid.

In yet another embodiment, a method of cooling detector components of a computed tomography (CT) detector is provided. The method includes controlling a liquid cooled thermal control to control a temperature of a cooling fluid at a predetermined temperature. The cooling fluid is cooled to the predetermined temperature with the liquid cooled thermal control. The cooling fluid is discharged into cooling channels positioned in thermal communication with a detector rail of a CT detector having a plurality of detector components. The cooling fluid in the cooling channels controls a temperature of the detector components at the predetermined temperature.

### BRIEF DESCRIPTION OF THE DRAWINGS

The presently disclosed subject matter will be better understood from reading the following description of non-limiting embodiments, with reference to the attached drawings, wherein below:

FIG. 1 is a schematic view of a liquid cooled thermal control system formed in accordance with an embodiment.

FIG. 2 is a top view of a detector rail formed in accordance with an embodiment.

FIG. 3 illustrates a schematic block diagram of the control system for a liquid cooled thermal control system formed in accordance with an embodiment.

FIG. 4 is a block diagram of a control module formed in accordance with an embodiment and configured to control a liquid cooled thermal control system.

FIG. 5 is a schematic block diagram of a liquid cooled thermal control system formed in accordance with another embodiment.

FIG. 6 is a schematic diagram of a liquid cooled thermal control system formed in accordance with an embodiment.

FIG. 7 illustrates graphs representative of the performance of liquid cooled thermal control systems formed in accordance with an embodiment.

FIG. 8 illustrates graphs representative of the performance of liquid cooled thermal control systems formed in accordance with other embodiments.

FIG. 9 illustrates graphs representative of the performance of a control system without an outer loop control.

FIG. 10 illustrates graphs representative of the performance of a control system with an outer loop control.

FIG. 11 illustrates a method for controlling a temperature of a computed tomography (CT) imaging system

FIG. 12 is a pictorial drawing of a computed tomography (CT) imaging system constructed in accordance with various embodiments.

FIG. 13 is a schematic block diagram of the CT imaging system of FIG. 12.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers, circuits or memories) may be implemented in a single piece of hardware or multiple pieces of hardware. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Although the embodiments are described with respect to a computed tomography (CT) detector, it should be noted that the liquid cooled thermal control described herein may be modified for use with other detectors or systems. For example, the liquid cooled thermal control may be utilized at least with a Positron Emission Tomography (PET) system, a Single Photon Emission Computed Tomography (SPECT) system, a Magnetic Resonance Imaging (MRI) system, and/or an X-ray system, among others. In one embodiment, the liquid cooled thermal control may be utilized with detectors formed from different materials.

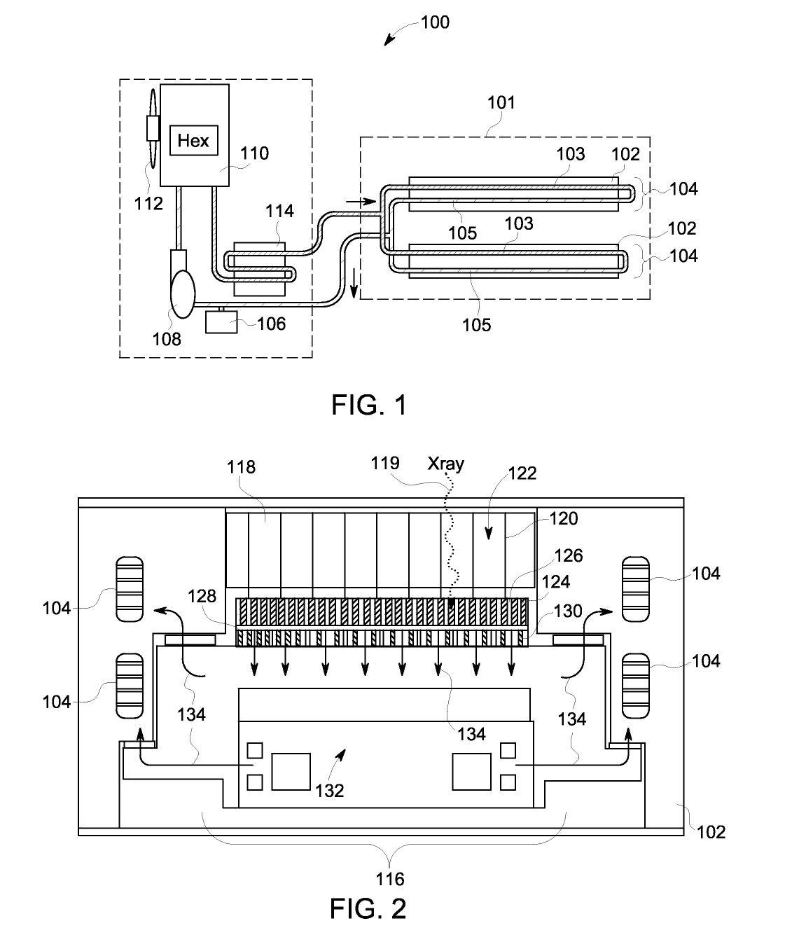

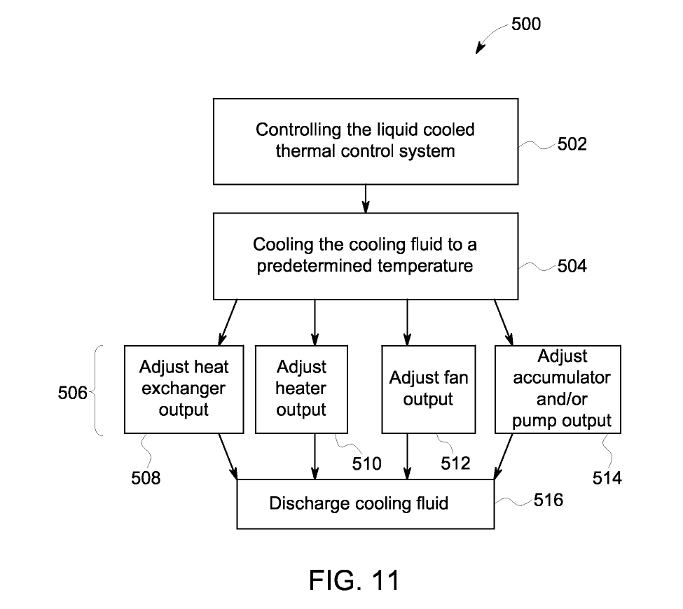

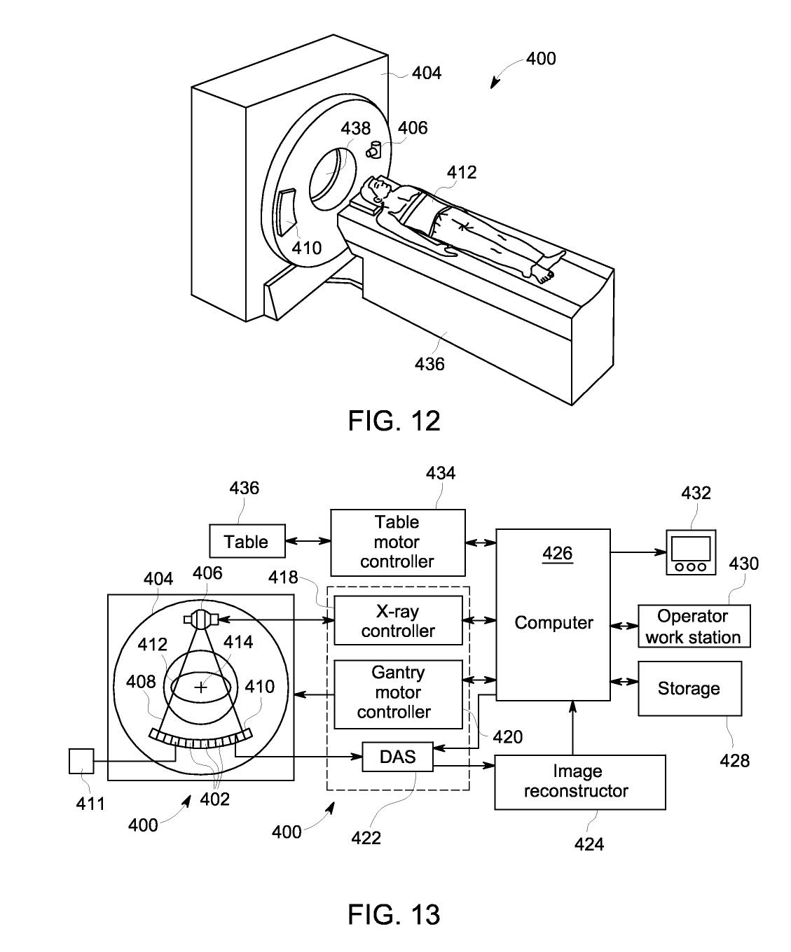

FIG. 1 is a schematic view of a liquid cooled thermal control system 100 for a CT detector 101, which may be embodied as the CT detector 400 shown in FIGS. 11 and 12. The thermal control system 100 is in thermal communication with detector rails 102 of the CT detector. In particular, cooling channels 104 of the thermal control system 100 are in thermal communication with the detector rails 102. The cooling channels 104 include a cool channel 103 and a hot channel 105. In one embodiment, the cooling channels 104 may extend through the detector rails 102. Alternatively, a cold plate (not shown) may be coupled to the detector rails 102. In such an embodiment, the cooling channels 104 may extend through the cold plate. Alternatively, the cooling channels 104 may be configured to extend both through the detector rails 102 and a cold plate. The cooling channels 104 have cooling fluid flowing therethrough, which may be any suitable cooling fluid (e.g. liquid or gas).

An accumulator 106 and a pump 108 are positioned downstream from the cooling channels 104. The accumulator 106 receives cooling fluid from the cooling channels 104. The amount of cooling fluid received in the accumulator 106 may depend on a pressure of the cooling fluid within the thermal control system 100, as described below. The pump 108 is positioned downstream of the accumulator 106 to control a flow of the cooling fluid thorough the thermal control system 100. The pump 108 may be a single speed pump or a variable speed pump.

The pump 108 discharges the cooling fluid downstream to a heat exchanger 110. The heat exchanger 110 may be any suitable heat exchanger, for example, an air-to-liquid heat exchanger or a liquid-to-liquid heat exchanger. In the illustrated embodiment, the heat exchanger 110 is an air-to-liquid heat exchanger having a fan 112. From the heat exchanger 110, the cooling fluid flows downstream to a heater 114. The heater 114 may be an electric heater, a gas heater, or any other suitable heater. The heater 114 discharges the cooling fluid downstream to the cooling channels 104.

During operation, the cool channels 103 receive the cooling fluid from the heater 114. The cooling fluid is provided at a predetermined temperature that is configured to maintain a temperature of the detector rails 102. The cooling fluid in the cooling channels 104 cools the detector rails 102 by receiving heat from the detector rails 102 through at least one of thermal induction or convection. The heated cooling fluid then flows through the hot channels 105 downstream to the accumulator 106. The accumulator 106 stores a portion of the cooling fluid based on a pressure within the thermal control system 100. For example, when the thermal control system 100 is operating at high pressures, the accumulator 106 may store more cooling fluid than when the system 100 is operating at low pressures. The accumulator 106 stores the cooling fluid to maintain a constant operating pressure of the thermal control system 100. The accumulator 106 accounts for expansion of the cooling fluid at high pressures and may be utilized to pressurize the pump 108, thereby, preventing cavitation within the pump 108.

The pump 108 receives cooling fluid from the accumulator 106. The pump 108 may be a variable speed pump that is controlled to adjust an amount of cooling fluid discharged to the heat exchanger 110. By controlling a speed of the pump 108, a temperature of the cooling fluid may be controlled. For example, increasing a speed of the pump 108 increases the liquid flow rate as the cooling fluid travels through the heat exchanger 110, which increases the cooling rate. Conversely, decreasing a speed of the pump 108 decreases the liquid flow rate as the cooling fluid flows through, the heat exchanger 110, which decreases the cooling rate. In one embodiment, the pump 108 discharges the cooling fluid to the heat exchanger 110 at rate configured to achieve the predetermined temperature of the cooling fluid.

In one embodiment, the heat exchanger 110 receives the cooling fluid from the pump 108. The heat exchanger 110 cools the cooling fluid to a temperature below the predetermined temperature. The fan 112 of the heat exchanger 110 may be controlled to adjust the temperature of the cooling fluid. For example, the fan 112 may be operated at a higher speed to increase the amount of cooling of the cooling fluid. Conversely, the fan 112 may be operated at a lower speed to decrease the amount of cooling of the cooling fluid. The speed of the fan 112 is controlled to achieve cooling of the cooling fluid to below the predetermined temperature.

The cooling fluid is discharged from the heat exchanger 110 downstream to the heater 114. The heater 114 heats the cooling fluid from below the predetermined temperature to the predetermined temperature. In particular, the heater 114 is capable of fine tuning the temperature of the cooling fluid, whereas, the heat exchanger 110 may not be capable of providing precise temperatures. Accordingly, the heat exchanger 110 is utilized to reduce the temperature of the cooling fluid to below the predetermined temperature. The heater 114 then fine tunes the temperature of the cooling fluid to achieve the predetermined temperature. The power supplied to the heater 114 may be controlled to adjust the temperature of the cooling fluid. By adjusting the power supplied to the heater 114, the heat produced by the heater is adjusted. For example, the heater 114 may be operated at a higher power to provide additional heating of the cooling fluid. Conversely, the heater 114 may be operated at a lower power to reduce an amount of heating of the cooling fluid. The heater 114 discharges the cooling fluid into the cool channels 103 at the predetermined temperature to maintain a temperature of the detector rails 102.

In various embodiments, the control system 100 is utilized to maintain a temperature of the detector rails 102 at a steady-state temperature. The control system 100 facilitates reducing or preventing changes in the temperature of the detector rails 102. The control system 100 may adjust several parameters to control the temperature of the cooling fluid. For example, any one of a speed of the pump 108, a speed of the fan 112, or a power of the heater 114 may be adjusted to achieve the predetermined temperature of the cooling fluid.

In one embodiment, the control system 100 may also be utilized to reduce a warm-up time of the CT detector. For example, the heat exchanger 110 may be shut-off and the heater 114 may be operated at a higher power to supply heated cooling fluid to the cooling channels 104. The heated cooling fluid may reduce the time required to warm-up the CT detector. In another embodiment, the heater 114 may be used to increase the dynamic range of air temperatures or gantry rotations to maintain the liquid temperature.

FIG. 2 is a top view of the detector rail 102 illustrating the components of the detector rail 102. One or more x-ray detectors 116 (one is shown) are positioned on the detector rail 102. The x-ray detector 116 includes a plurality of detector components. A collimator 118 is provided to direct or collimate x-rays 119 emitted from a subject. The collimator 118 includes a plurality of plates 120, for example, tungsten plates that define openings 122 therebetween. The openings 122 are configured to direct the x-rays 119 to a scintillator 124. The x-ray detector 116 is not limited to including the scintillator 124. In other embodiments, the x-ray detector 116 may include other detector materials, for example, a direct conversion material. The scintillator 124 includes openings 126 in a pixel configuration. The openings 126 of the scintillator 124 are aligned with the openings 122 of the collimator 118. The scintillator 124 detects the x-rays 119 at different pixel locations and directs the x-rays 119 to a plurality of photodiodes 128. The scintillator 124 convert the x-rays 119 into light waves. The photodiodes 128 covert the light into electrical charge (e.g., electrical signals) that is converted to digital signals with an analog-to-digital (A/D) converter 130. The digital signals may be used to generate an image of the subject. Electronic components 132 receive the digital signals from the A/D converter 130 and process the digital signals to generate the image.

In the illustrated embodiment, the cooling channels 104 extend through the detector rail 102. The cooling channels 104 are in thermal contact with and receive heat from the detector rail 102 to maintain the detector rail 102 at a constant or nearly constant temperature, such as within a tolerance or variance range The cooling channels 104 may maintain a temperature of the detector rails 102 within a range for normal detector operation. In particular, if the temperature of the detector rail 102 changes during operation, the detector rail 102 may contract and/or expand. Contraction and/or expansion of the detector rail 102 may result in shifting of the detector components. For example, the collimator 118 and the scintillator 124 may shift, causing the openings 122 of the collimator 118 to become misaligned with the openings 126 of the scintillator 124. Such misalignment may result in scatter and/or noise in the image data. The cooling channels 104 maintain a temperature of the detector rail 102 to reduce the amount of or to prevent contraction and/or expansion of the detector rail 102, thereby reducing or preventing shifting of the detector components. As such, the cooling channels 104 facilitate maintaining alignment of the openings 122 of the collimator 118 and the openings 126 of the scintillator 124.

The cooling channels 104 are also configured to receive heat 134 from the x-ray detector 116. The cooling channels 104 are in thermal contact with and receive heat from the x-ray detector 116 to maintain a constant or nearly constant temperature of the detector components. In particular, some components, for example, the photodiodes 128 may be sensitive to changes in temperature. Changes in temperature may cause the photodiodes 128 to become damaged and/or malfunction. The cooling channels 104 receive heat through thermal induction or convection from the x-ray detector 116 to maintain a temperature of the photodiodes 128 and other detector components to reduce the likelihood of or prevent damage to and/or malfunctioning of the components.

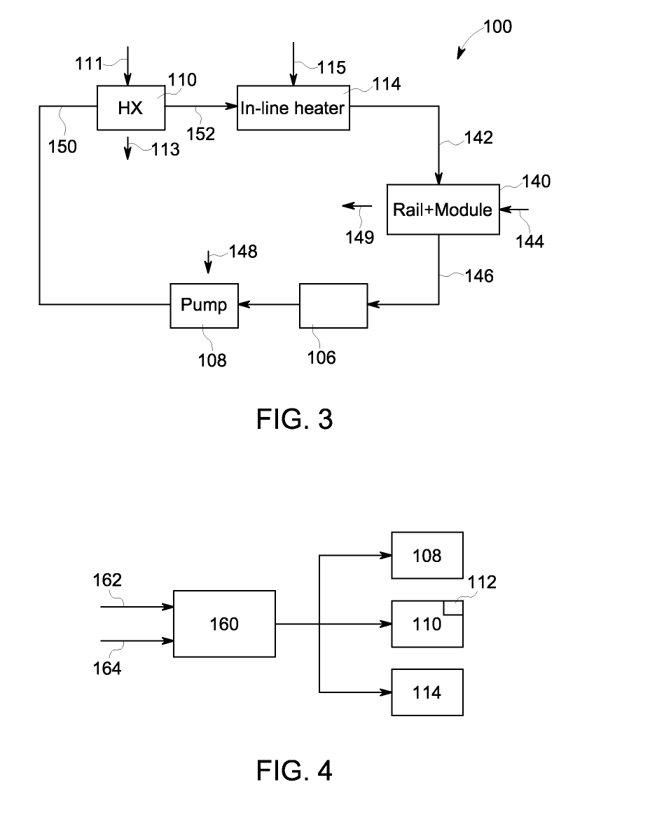

FIG. 3 illustrates a schematic block diagram of the control system 100. The detector rail 102 and the x-ray detector 116 (both shown in FIG. 2) are illustrated as a module 140. The module 140 receives input cooling fluid 142 at the predetermined temperature. Additionally, the module 140 receives a heat load 144 from the detector components and/or the gantry, for example. Heat from the heat load 144 is transferred to the cooling fluid to produce output cooling fluid 146 having a temperature that is greater than the temperature of the input cooling fluid 142. Heat loss 149 is discharged from the module 140 as the cooling fluid flows through the accumulator 106 and the pump 108. The pump 108 is operated based on a flow rate control signal 148 that is selected to control a temperature of the cooling fluid.

The cooling fluid flows downstream to the heat exchanger 110 and enters the heat exchanger 110 at an input 150 at a temperature that is greater than the predetermined temperature. The heat exchanger 110 operates at a fan speed, for example, based on a fan speed control signal 111, to reduce the temperature of the cooling fluid to an output 152 at a temperature that is below the predetermined temperature. The cooling fluid then travels downstream to the heater 114. The cooling fluid enters the heater 114 at or about at the temperature of the fluid at the output 152. The heater 114 is operated at a power level that defines a heating level, for example, based on a heat control signal 115, to heat the cooling fluid to the predetermined temperature. The heater 114 discharges the cooling fluid to the module 140 as input cooling fluid 142. The flow rate control signal 148 of the pump 108, the fan speed control signal 111 of the heat exchanger 110, and/or the heat control signal 115 of the heater 114 may be adjusted to control a temperature of the input cooling fluid 142.

FIG. 4 is a block diagram of a control module 160 formed in accordance with an embodiment and configured to control the liquid cooled thermal control system 100. The control module 160 may be hardware, software or a combination thereof configured to provide instructions to the control system 100, such as the various control signals described herein. The control module 160 may be configured to operate software to provide instructions to the control system 100. The software may be a tangible and non-transitory machine-readable medium or media having instructions recorded thereon for the processor to operate the control system 100. The medium or media may be any type of CD-ROM, DVD, hard disk, optical disk, flash RAM drive, or other type of computer-readable medium or a combination thereof.

The control module 160 is in communication with the pump 108, the fan 112 of the heat exchanger 110, and the heater 114. The control module 160 is configured to control the operation of any one or more of the pump 108, the fan 112, or the heater 114. For example, the control module 160 may control a speed of the pump 108, a speed of the fan 112, and/or a power level of the heater 114. The control module 160 receives a temperature input signal 162 indicative of the temperature of at least one of the detector rail 102 or the x-ray detector 116 (both shown in FIG. 2). The control module 160 compares the temperature input signal 162 to a temperature setpoint 164, which may be predetermined. The temperature setpoint 164 is indicative of a desired or required predetermined temperature of the detector rail 102 or a desired or required predetermined temperature of the x-ray detector 116. The temperature setpoint 164 may be entered, for example, by an operator prior to operation of the CT detector.

The control module 160 determines a difference between the temperature input signal 162 and the temperature setpoint 164 to determine adjustments to the control system 100. For example, the control module 160 adjusts the operation of the control system 100 to achieve a temperature based on the temperature input signal 162 (which may be a feedback signal) that is substantially equivalent to the temperature setpoint 164. For example, the control module 160 may adjust a speed of the fan 112, a speed of the pump 108, a power level of the heater 114, or any combination thereof to achieve a temperature level that is substantially equivalent or equal to the temperature setpoint 164.

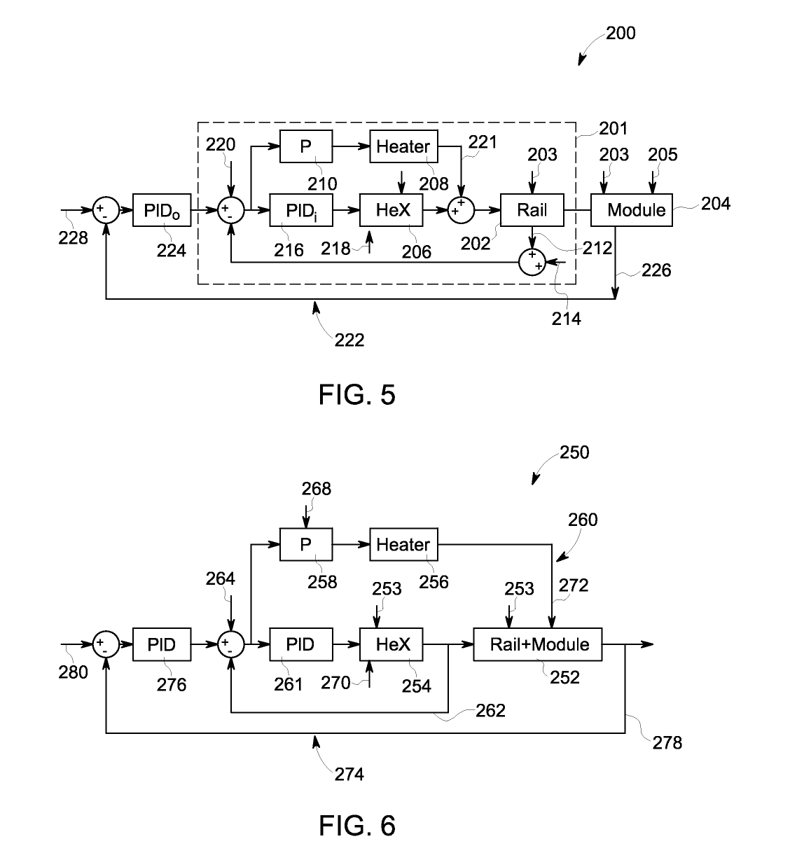

FIG. 5 is a schematic diagram of a liquid cooled thermal control system 200 formed in accordance with another embodiment. The control system 200 is in fluid communication with a detector rail 202 and an x-ray detector 204. The detector rail 202 and the x-ray detector 204 both receive a heat load 203 from a gantry of the CT detector. The x-ray detector 204 also may generate a heat load 205. The control system 200 includes a heat exchanger 206, an in-line heater 208, and a pump 210. In operation, cooling fluid passes through the detector rail 202 to cool the detector rail 202 and the x-ray detector 204. The control system 200 includes an inner control loop 201. A detector rail temperature signal 212 and a pump flow rate signal 214 are delivered to a control module 216. The control module 216 adjusts a fan speed 218 of the heat exchanger 206 based on a comparison of at least one of the detector rail temperature signal 212 and the pump flow rate signal 214 to a cooling fluid temperature setpoint 220, which may be predetermined. Alternatively or additionally, the pump flow rate signal 214 may be adjusted by the control module 216 based on the comparison of at least one of the detector rail temperature signal 212 and the pump flow rate signal 214 to the cooling fluid temperature setpoint 220. Alternatively or additionally, a power level signal 221 of the heater 208 may be adjusted by the control module 216 based on the comparison of at least one of the detector rail temperature signal 212 and the pump flow rate signal 214 to the cooling fluid temperature setpoint 220.

The control system 200 also includes an outer control loop 222. The outer control loop 222 includes a control module 224 that receives a temperature input 226 (e.g. measured temperature or temperature signal) from the x-ray detector 204. The control module 224 also receives an x-ray detector temperature setpoint 228. Based on a comparison of the temperature input 226 and the temperature setpoint 228, the control module 224 may adjust the cooling fluid temperature setpoint 220. Accordingly, the control system 200 includes two feedback loops capable of adjusting at least one of the pump flow rate 214, the fan speed 218 of the heat exchanger 206, or the heater output 221 to control a temperature of the cooling fluid. The inner control loop 201 and the outer control loop 222 may operate independently or separately.

FIG. 6 is a schematic diagram of a liquid cooled thermal control system 250 formed in accordance with another embodiment. The control system 250 is in fluid communication with a detector module 252 including a detector rail and an x-ray detector. The control system 250 includes a heat exchanger 254, a surface heater 256, and a pump 258. The heat exchanger 254 and the detector module 252 both receive a heat load 253 from a gantry of the CT detector. The control system 250 includes an inner control loop 260 having a control module 261. The inner control module 261 receives a temperature input 262 from the heat exchanger 254. The temperature input 262 is compared to a cooling fluid temperature setpoint 264 by the control module 261. Based on the comparison, the control module 261 may adjust at least one of a pump flow rate 268 of the pump 258, a fan speed 270 of the heat exchanger 254, or a heater output 272 of the heater 256 to control a temperature of the cooling fluid.

The control system 250 also includes an outer control loop 274 having an outer control module 276. A control module 276 receives a detector module temperature input 278 (e.g. measured temperature or temperature signal) and a detector module temperature setpoint 280. Based on a comparison of the detector module temperature input 278 and the detector module temperature setpoint 280, the control module 276 may adjust the cooling fluid temperature setpoint 264. Accordingly, the control system 250 includes two feedback loops capable of adjusting at least one of the pump flow rate 268 of the pump 258, the fan speed 270 of the heat exchanger 254, or the heater output 272 of the heater 256 to control a temperature of the cooling fluid. The inner control loop 260 and the outer control loop 274 may operate independently or separately.

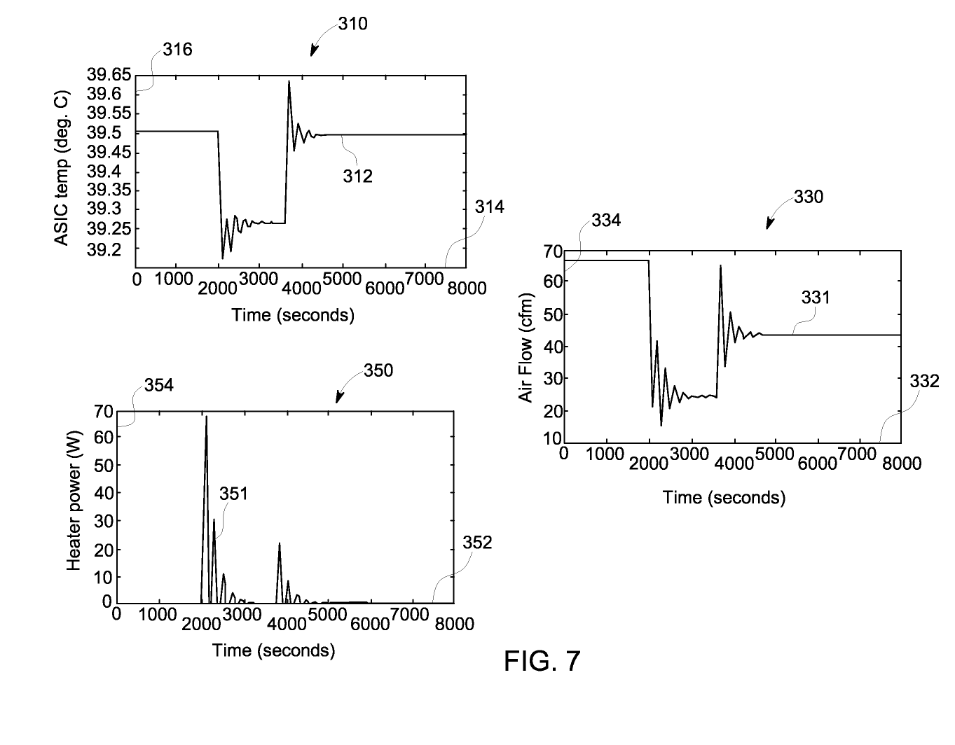

FIG. 7 illustrates graphs representative of the performance of the control system 200. Graph 310 illustrates a temperature 312 of an A/D converter, for example, the A/D converter 130 (shown in FIG. 2), cooled by the control system 200. The x-axis 314 illustrates time in seconds and the y-axis 316 illustrates temperature in degrees Celsius. Graph 330 illustrates an airflow 331 through the heat exchanger 206 of the control system 200. The x-axis 332 represents time in seconds and the y-axis 334 represents airflow in cubic feet per minute. Graph 350 illustrates a heater power 351 of the heater 208 of the control system 200. The x-axis 352 illustrates time in seconds and the y-axis 354 illustrates heater power in Watts. In the illustrated embodiment, the heater power is below 70 W.

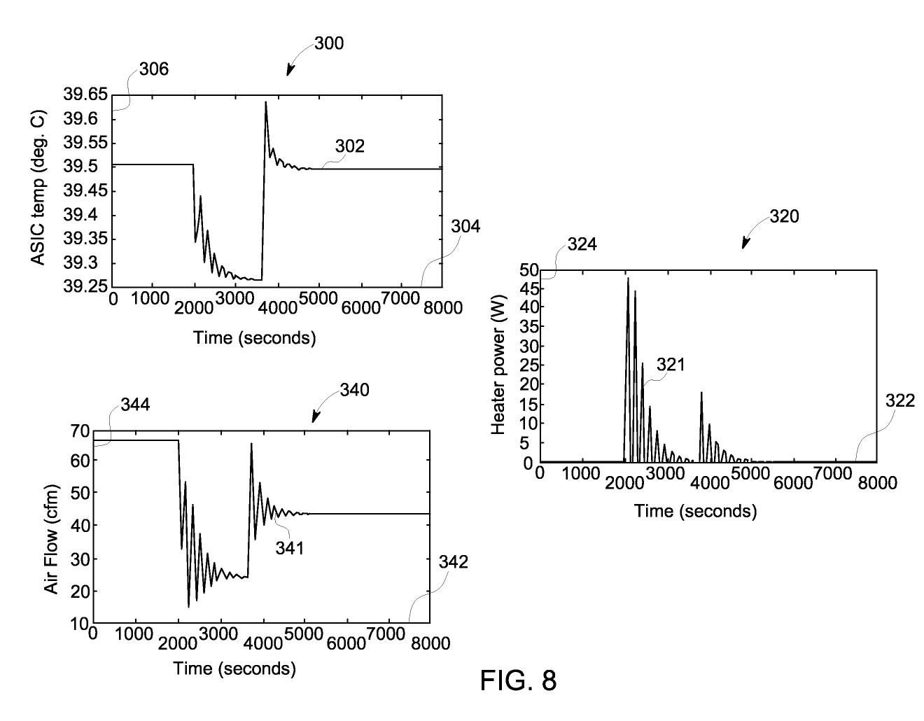

FIG. 8 illustrates graphs representative of the performance of the control system 250. Graph 300 illustrates a temperature 302 of an A/D converter cooled by the control system 250. The x-axis 304 illustrates time in seconds and the y-axis 306 illustrates temperature in degrees Celsius. Graph 320 illustrates airflow 321 through the heat exchanger 254 of the control system 250. The x-axis 322 represents time in seconds and the y-axis 324 represents airflow in cubic feet per minute. Graph 340 illustrates a heater power 341 of the heater 256 of the control system 250. The x-axis 342 illustrates time in seconds and the y-axis 344 illustrates heater power in Watts. In the illustrated embodiment, the heater power is below 50 W.

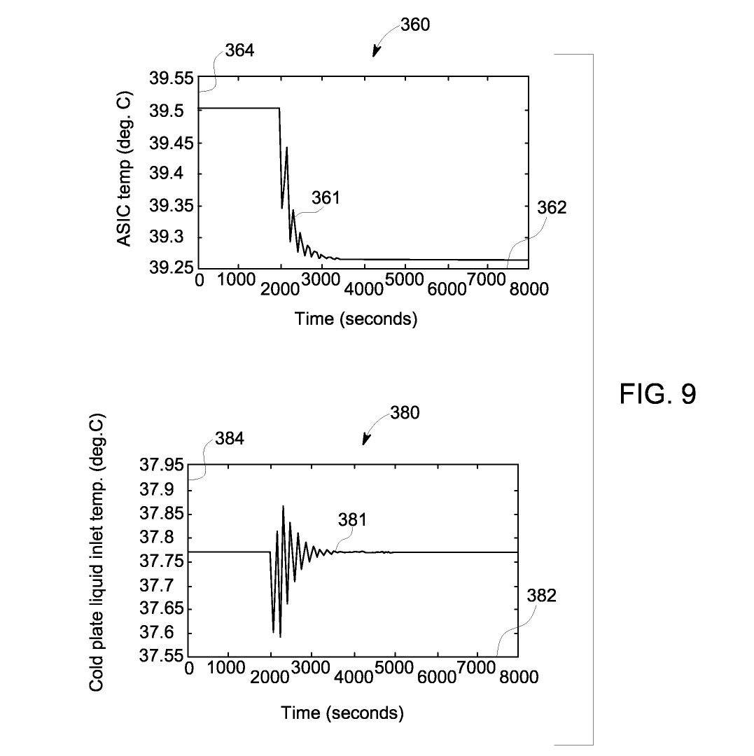

FIG. 9 illustrates graphs representative of the performance of a control system without an outer loop control. Graph 360 illustrates a temperature 361 of an A/D converter used with a control system having no outer control loop. The x-axis 362 represents time in seconds and the y-axis 364 represents temperature. Graph 380 illustrates an inlet temperature 381 of cooling fluid in cooling channels used in a control system having no outer control loop. The x-axis 382 represents time in seconds and the y-axis 384 represents temperature.

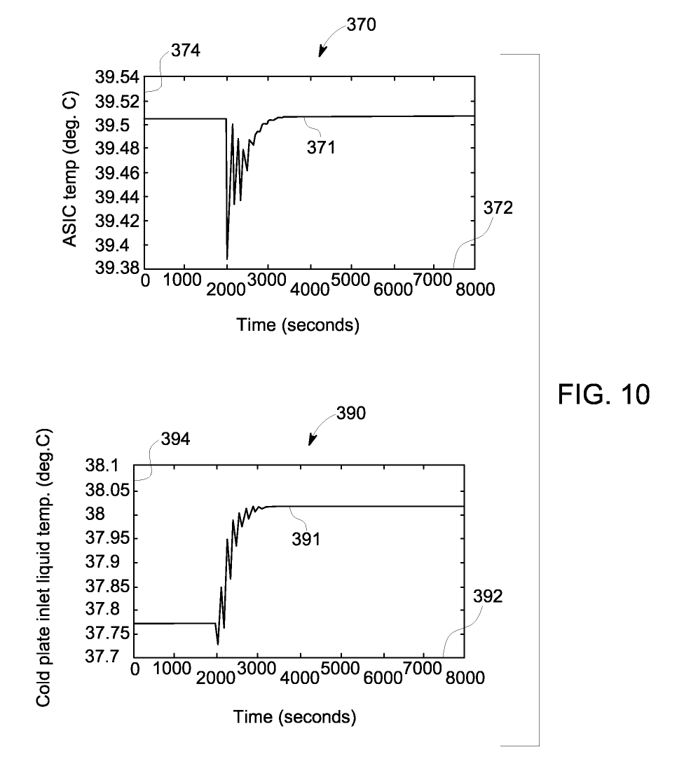

FIG. 10 illustrates graphs representative of the performance of a control system with an outer loop control. Graph 370 illustrates a temperature 371 of an A/D converter used with a control system having an outer control loop. The x-axis 372 represents time in seconds and the y-axis 374 represents temperature. Graph 390 illustrates an inlet temperature 391 of cooling fluid in cooling channels used in a control system having an outer control loop. The x-axis 392 represents time in seconds and the y-axis 394 represents temperature.

As illustrated in FIGS. 9 and 10, various embodiments of a control system with one or more outer loops adjusts the liquid temperature to maintain a constant detector electronics (e.g., photodiode) temperature with adjustment of a liquid set-point based on changes in the temperature of the detector electronics. In various embodiments, this scheme may compensate for changes in the heat load of the detector electronics and the impact of rotation on rails by convection. A control system without one or more outer loops that adjusts the liquid temperature does not compensate for heat load changes of the detector electronics and the impact of gantry rotation on detector cooling, which is accomplished by a design that keeps the power of the electronics at a constant value and by providing insulated rails to reduce the impact of gantry rotation on the temperature of the detector electronics.

FIG. 11 illustrates a method 500 for controlling a temperature of a computed tomography (CT) imaging system. The method includes controlling 502 a liquid cooled thermal control system to control a temperature of a cooling fluid at a predetermined temperature. The liquid cooled thermal control system may include a heat exchanger, a heater, or a fan. An output of at least one of the heat exchanger, the heater, or the fan may be controlled in response to disturbances in the CT imaging system. For example, the disturbances may include a gantry air temperature and/or heat convection generated by rotating the gantry. The disturbances may also include heat generated by detector components within the CT system. For example, an analog-to-digital converter may generate heat within the CT system.

The method 500 further includes cooling 504 the cooling fluid to the predetermined temperature with the liquid cooled thermal control system. A control module may adjust 506 parameters of the liquid cooled thermal control system in response to the disturbances. For example, in one embodiment, a control module of the liquid cooled thermal control system may adjust 508 an output of the heat exchanger. In another embodiment, the liquid cooled thermal control system may adjust 510 an output of the heater. In yet another embodiment, the liquid cooled thermal control system may adjust 512 an output of the fan. Moreover, the liquid cooled thermal control system may adjust 514 an output of at least one of an accumulator or a pump. In an exemplary embodiment, the liquid cooled thermal control system may carry out any combination of the adjustment steps 508, 510, 512, and/or 514.

The method 500 also includes discharging 516 the cooling fluid into cooling channels positioned in thermal communication with a detector rail of a CT detector having a plurality of detector components. The cooling fluid in the cooling channels controls a temperature of the detector components at the predetermined temperature.

In one embodiment, an initial conductance of the heat exchanger is defined as:

G=Total Heat load/ITD=Qₜₒₜₐₗ/(T_{liq-hot}−Tₐᵢᵣ)

By Varying an air flow rate (e.g. varying the cubic feet per minute of airflow by adjusting a fan speed within the liquid cooled thermal control system) the conductance may be varied to control a liquid temperature for various air temperature conditions within the gantry. For a heat exchanger design the initial conductance is function of air flow rate (CFM) and liquid flow rate (GPM). Using these two variables, the outlet liquid temperature of the heat exchanger can be controlled. A fan speed control in the heat exchanger may reduce an error from a set-point.

An inline heater may be used in a control loop to fine tune the control of liquid temperature. A heater power may be manipulated to fine tune the liquid temperature outlet at the inline heater that is fed to the detector. In some embodiments, a power could be variable with a convection boundary condition changing along with air temperature changes. In such embodiments, a cascade loop is incorporated, where an inner loop controls the liquid inlet temperature to the detector using the fan, and/or the pump and/or the inline heater while an outer loop feedbacks the detector module temperature and thereby resets the inner loop liquid temperature for control.

Referring to FIGS. 12 and 13, a multi-slice scanning imaging system, for example, a CT imaging system 400 is shown as including a plurality of the detectors 402 and in which the various embodiments may be implemented. The system 400 may be used with the liquid cooled thermal control systems described above. The CT imaging system 400 includes a gantry 404, which includes an x-ray source 406 (also referred to as an x-ray source 406 herein) that projects a beam of x-rays 408 toward a detector array 410 on the opposite side of the gantry 404. A cooling system 411, for example, any of the cooling systems described above, is in thermal contact with the detector array 410. Alternatively, the cooling system 411 may be in thermal contact with any of the components of the CT imaging system 400. The detector array 410 is formed by a plurality of detector rows (not shown) including a plurality of the detectors 402 that together sense the projected x-rays that pass through an object, such as a medical patient 412 between the array 410 and the source 406. Each detector 402 produces an electrical signal that represents the intensity of an impinging x-ray beam and hence can be used to estimate the attenuation of the beam as the beam passes through the patient 412. During a scan to acquire x-ray projection data, the gantry 404 and the components mounted therein rotate about a center of rotation 414. FIG. 7 shows only a single row of detectors 402 (i.e., a detector row). However, the multi-slice detector array 410 includes a plurality of parallel detector rows of detectors 402 such that projection data corresponding to a plurality of quasi-parallel or parallel slices can be acquired simultaneously during a scan.

Rotation of components on the gantry 404 and the operation of the x-ray source 406 are controlled by a control mechanism 416 of the CT imaging system 400. The control mechanism 416 includes an x-ray controller 418 that provides power and timing signals to the x-ray source 406 and a gantry motor controller 420 that controls the rotational speed and position of components on the gantry 404. A data acquisition system (DAS) 422 in the control mechanism 416 samples analog data from the detectors 402 and converts the data to digital signals for subsequent processing. An image reconstructor 424 receives sampled and digitized x-ray data from the DAS 422 and performs high-speed image reconstruction. The reconstructed image is applied as an input to a computer 426 that stores the image in a storage device 428. The image reconstructor 424 can be specialized hardware or computer programs executing on the computer 426.

The computer 426 also receives commands and scanning parameters from an operator via a console 430 that has a keyboard and/or other user input and/or marking devices, such as a mouse, trackball, or light pen. An associated display 432, examples of which include a cathode ray tube (CRT) display, liquid crystal display (LCD), or plasma display, allows the operator to observe the reconstructed image and other data from the computer 426. The display 432 may include a user pointing device, such as a pressure-sensitive input screen. The operator supplied commands and parameters are used by the computer 426 to provide control signals and information to the DAS 422, x-ray controller 418, and gantry motor controller 420. In addition, the computer 426 operates a table motor controller 434 that controls a motorized table 436 to position the patient 412 in the gantry 404. For example, the table 436 moves portions of the patient 412 through a gantry opening 438.

Various embodiments provide a thermal control system that may be mounted to and receives heat from detector rails and/or cold plates to receive heat from the detector components. The thermal control system has a controlled temperature (e.g. substantially constant temperatures) cooling fluid circulating therethrough to maintain the detector rails at constant temperature, for example, in response to one or more disturbances that fluctuates or changes a temperature of the detector rails or an x-ray detector coupled to the detector rails. The cooling fluid temperature is controlled in various embodiments using a heat exchanger, a heater, and a pump that act as actuators for temperature control. A fan speed of the heat exchanger may be controlled using a control module based on error in the cooling fluid temperature required and a measured cooling fluid temperature. The heater power also may be modulated to control the cooling fluid temperature supplied to the detector rails. A pump speed also may be controlled to achieve a required cooling fluid flow rate through the thermal control system.

In various embodiments, the control module parameters are calculated based on a difference in cooling fluid temperature and air temperature to account for the gain differences required to achieve temperature control. In one embodiment, a feedback in a cascade outer loop is provided to change a cooling fluid temperature setpoint to compensate for heat load changes in the detector components. Alternately, a surface heater may be mounted to the cold plate and/or detector rails. A power of the heater is modulated to control the rail temperature where the thermal control system is mounted.

At least one technical effect of some embodiments is maintaining a constant detector electronics temperature.

Various embodiments described herein provide a tangible and non-transitory machine-readable medium or media having instructions recorded thereon for a processor or computer to operate an imaging apparatus to perform an embodiment of a method described herein. The medium or media may be any type of CD-ROM, DVD, floppy disk, hard disk, optical disk, flash RAM drive, or other type of computer-readable medium or a combination thereof.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the described subject matter without departing from their scope. While the dimensions and types of materials described herein are intended to define the parameters of the various embodiments of the invention, the embodiments are by no means limiting and are exemplary embodiments. Many other embodiments will be apparent to one of ordinary skill in the art upon reviewing the above description. The scope of the various embodiments of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “Wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable one of ordinary skill in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A computed tomography (CT) detector comprising:
a detector rail;
an x-ray detector positioned on the detector rail, the x-ray detector including a plurality of detector components, at least some of the detector components configured to detect x-rays;
a liquid cooled thermal control system having cooling channels in thermal communication with the detector rail, the cooling channels having a cooling fluid flowing therethrough to control a temperature of the detector components in response to one or more disturbances that changes a temperature of the x-ray detector; and
a control module provided in the liquid cooled thermal control system to adjust parameters of the liquid cooled thermal control system in response to the disturbances.

2. The CT detector of claim 1 further comprising a gantry, the detector rail positioned within the gantry, the disturbances including an air temperature of the gantry and heat convection generated by rotation of the gantry.

3. The CT detector of claim 1, wherein the control adjusts parameters of the liquid cooled thermal control system so that cooling fluid flowing through the cooling channels maintains a temperature of the detector components at a level for normal detector operation.

4. The CT detector of claim 1, wherein the detector components include a plurality of photodiodes and an analog-to-digital converter positioned adjacent to the photodiodes, the disturbances including heat generated by the analog-to-digital converter, the heat received by the photodiodes, the control adjusting parameters of the liquid cooled thermal control system so that the cooling channels control a temperature of the photodiodes.

5. The CT detector of claim 1, wherein the detector components include a collimator and a scintillator, the collimator having openings aligned with openings of the scintillator, the control adjusting parameters of the liquid cooled thermal control system so that the cooling channels control a temperature of the detector rail to maintain an alignment of the openings of the collimator with the openings of the scintillator.

6. The CT detector of claim 1, wherein the control adjust parameters of the liquid cooled thermal control system to maintain a steady-state temperature of the detector components.

7. The CT detector of claim 1, wherein the liquid cooled thermal control system includes a heat exchanger in fluid communication with the cooling channels, the control adjusting an output of the heat exchanger to control cooling of the cooling fluid.

8. The CT detector of claim 1, wherein the liquid cooled thermal control system further comprises a heat exchanger and a heater in fluid communication with the cooling channels, the control adjusting an output of the heat exchanger to cool the cooling fluid to below a predetermined temperature, the control controlling an output of the heater to raise the temperature of the cooling fluid from below the predetermined temperature to the predetermined temperature.

9. The CT detector of claim 1, wherein the liquid cooled thermal control system further comprises a heater in fluid communication with the cooling channels, the control adjusting the output of the heater to heat the cooling fluid in the cooling channels to heat the CT detector.

10. A liquid cooled thermal control system for a computed tomography (CT) detector comprising:
cooling channels in thermal communication with a detector rail of the CT detector, the cooling channels having a cooling fluid flowing therethrough to control a temperature of detector components positioned on the detector rail in response to one or more disturbances that changes a temperature of the detector rail;
a heat exchanger for receiving heated cooling fluid from the cooling channels, the heat exchanger cooling the cooling fluid;
a heater for receiving the cooled cooling fluid from the heat exchanger, the heater heating the cooled cooling fluid from the heat exchanger and discharging the cooling fluid into the cooling channels; and
a control module for controlling at least one of the heat exchanger, the heater, or a fan of the heat exchanger to control a temperature of the cooling fluid.

11. The control system of claim 10 further comprising a pump to control a flow of the cooling fluid from the cooling channels to the heat exchanger, the control module controlling a speed of the pump.

12. The control system of claim 10, wherein:
the heat exchanger cools the cooling fluid to below a predetermined temperature; and
the heater raises the temperature of the cooling fluid from below the predetermined temperature to the predetermined temperature.

13. The control system of claim 10, wherein the heater heats the cooling fluid discharged into the cooling channels to heat the CT detector.

14. The control system of claim 10 further comprising an accumulator positioned upstream of the heat exchanger and downstream of the cooling channels, the control module controlling the accumulator to control a pressure of the cooling fluid.

15. The control system of claim 10 further comprising a gantry, the detector rail positioned within the gantry, the disturbances including an air temperature of the gantry and heat convection generated by rotation of the gantry.

16. A method of cooling detector components of a computed tomography (CT) detector comprising:
controlling a liquid cooled thermal control system to control a temperature of a cooling fluid at a predetermined temperature;
cooling the cooling fluid to the predetermined temperature with the liquid cooled thermal control system; and
discharging the cooling fluid into cooling channels positioned in thermal communication with a detector rail of a CT detector having a plurality of detector components, the cooling fluid in the cooling channels controlling a temperature of the detector components at the predetermined temperature.

17. The method of claim 16, wherein the detector components include a plurality of photodiodes and an analog-to-digital positioned adjacent to the photodiodes and generating heat that is received by the photodiodes, the method further comprising controlling a temperature of the photodiodes with the cooling fluid discharged into the cooling channels.

18. The method of claim 16, wherein the detector components include a collimator and a scintillator, the collimator having openings aligned with openings of the scintillator, the method further comprising controlling a temperature of the detector rail with the cooling fluid discharged into the cooling channels to maintain an alignment of the openings of the collimator with the openings of the scintillator.

19. The method of claim 16, wherein the liquid cooled thermal control system includes a heat exchanger and a heater in fluid communication with the cooling channels, the method further comprising:
cooling the cooling fluid flowing with the heat exchanger to below a predetermined temperature; and
raising the temperature of the cooling fluid from below the predetermined temperature to the predetermined temperature with the heater.

20. The method of claim 16, wherein the liquid cooled thermal control system includes a heater in fluid communication with the cooling channels, the method further comprising heating the cooling fluid with the heater to heat the CT detector.
